(19) United States
(12) Patent Application Publication
(10) Pub. No.: US 2017/0273629 A1
(43) Pub. Date: Sep. 28, 2017

(54) HEALTH MONITORING TELEVISION

(71) Applicants: BOE TECHNOLOGY GROUP CO., LTD., Beijing (CN); BEIJING BOE MULTIMEDIA TECHNOLOGY CO., LTD., Beijing (CN)

(72) Inventors: Shuhuan YU, Beijing (CN); Junning SU, Beijing (CN); Peng CHENG, Beijing (CN); Jinglu BAI, Beijing (CN)

(21) Appl. No.: 15/512,400

(22) PCT Filed: Jan. 20, 2016
(86) PCT No.: PCT/CN2016/071451

§ 371 Date: Mar. 17, 2017

(30) Foreign Application Priority Data
Aug. 24, 2015 (CN) 201510521176.6

(51) Int. Cl. A61B 5/00; A61B 5/0205; H04N 21/4223; H04N 21/422; H04N 21/442; H04N 21/4415; A61B 5/1171; A61B 5/0402

(52) U.S. Cl. CPC A61B 5/6891; A61B 5/0017; A61B 5/0077; A61B 5/1176; A61B 5/749; A61B 5/02055; A61B 5/0402; A61B 5/7282; A61B 5/7445; H04N 21/42201; H04N 21/44213; H04N 21/4415; H04N 21/4223; A61B 5/021

(57) ABSTRACT

The present disclosure relates to a health monitoring television, comprising: a physiological parameter acquisition device for acquiring a physiological parameter of a monitored object; a central processor for generating health monitoring information based on the acquired physiological parameter; and an information exhibition device for outputting the health monitoring information to a user. The health monitoring television according to the present disclosure endows the commonly used television in ordinary families with the health monitoring function, i.e., adding the central processor and the physiological parameter acquisition device, so as to enable the monitored object, particularly the old people, to learn their own physical status easily through direct visual and/or audible information.

### RELATED APPLICATIONS

The present application is the U.S. national phase entry of PCT/CN2016/071451, with an international filing date of Jan. 20, 2016, which claims the benefit of Chinese Patent Application No. 201510521176.6, filed on Aug. 24, 2015, the entire disclosures of which are incorporated herein by reference.

### FIELD OF THE INVENTION

The present disclosure relates to the field of health monitoring, particularly to a health monitoring television.

### BACKGROUND OF THE INVENTION

In today's society, the aging process is accelerated, and the aged suffer from all kinds of diseases. However, the young people have to go out for work due to increasing life pressure and could not look after the aged often. Therefore, people begin to pay more and more attention to the situation that accident occurs when the aged stay at home alone. In order to prevent occurrence of such accident, a home monitoring with respect to the physical status of the aged has been extremely urgent. In addition, many chronic patients also need a long term home monitoring. Moreover, with economic development and social progress, people have higher and higher requirements on quality of life and are more and more aware about the health conditions, hence, daily health care and health monitoring have been becoming an essential part of many people's lives. Particularly, it is more necessary to perform health monitoring on people such as pregnant women, infants, and people in sub-health state.

At present, there are generally two kinds of devices for health monitoring of human body. One is the conventional medical monitoring instruments, such as the various monitoring instruments commonly used for body organs like heart, brain etc. in sickrooms of the hospital; and the other is a variety of emerging portable smart devices such as wearable devices and smart mobile phones for monitoring physiological parameters of a person. All the above mentioned devices have certain shortcomings for home monitoring. The former belongs to the professional devices used in hospital, of which the wiring is messy and the operation is complex, and thus it can only be operated by or used under direction of medical staff after professional learning or training generally, while it is difficult for the ordinary person to operate and use it individually. Moreover, the cost of professional device is relatively high and it is unaffordable for ordinary families. In addition, it seems to someone that such medical monitoring instruments intuitively are dedicated to critically ill patients, which may easily result in mental pressure and nervous emotion to the monitored object so as to possibly influence accuracy of the monitoring data to some extent. Although the latter has certain portability, these smart devices (such as a smart mobile phone) generally have redundant functions and complex operations and thus are extremely inconvenient to use for ordinary people, particularly the aged or patients with impaired function.

### SUMMARY OF THE INVENTION

An object of the present disclosure is to provide a health monitoring television that can overcome the above defects.

The present disclosure makes use of the television that is required in ordinary families to make appropriate improvements so as to combine the human body health monitoring function into the ordinary television in the case of not influencing the conventional function of the television, thereby obtaining a television with the health monitoring function. Specifically, the main principle of the present disclosure lies in adding a physiological parameter acquisition device (such as a heart rate sensor, a blood pressure sensor etc.) and a central processor on the basis of the ordinary television. The former is used for acquiring various physiological parameters of the human body, while the latter is used for processing the acquired physiological parameter and based thereon generating health monitoring information, meanwhile, controlling a output device of the television (including video and audio output devices, such as television screen and speaker) to exhibit the health monitoring information to the user directly, so as to enable the user to learn his own physical status easily, which enhances the user experience. Further, a network communication function can also be added to transmit the resulting health monitoring information to a database in the network server, so as to enable other persons (such as doctor or family member) to learn the physical status of the monitored object at home in real time through the networking terminal (mobile phone or computer etc.). In addition, the health monitoring information can comprise processing suggestions with respect to the corresponding physical status, so as to enable the user to adopt appropriate countermeasures calmly when facing emergency to avoid further deterioration.

According to one aspect of the present disclosure, there is proposed a health monitoring television comprising: a physiological parameter acquisition device for acquiring a physiological parameter of a monitored object; a central processor for generating health monitoring information based on the acquired physiological parameter; and an information exhibition device for outputting the health monitoring information to a user. The information exhibition device can comprise at least one of a television screen and a television speaker, and the health monitoring information can comprise the acquired physiological parameter.

Apparently, by using the health monitoring television according to the present disclosure to perform family health monitoring, the following beneficial effects can be achieved: endowing the commonly used television in ordinary families with the health monitoring function, i.e., adding the central processor and the physiological parameter acquisition device, enables the monitored objects, such as the patients or the aged, to learn their own physical status easily through direct visual and/or audible information.

In one embodiment of the health monitoring television according to the present disclosure, the health monitoring television may further comprise a memory for storing a normal physiological parameter value corresponding to the acquired physiological parameter for comparison with the actually acquired physiological parameter. In addition, the memory can be further used for storing the acquired physiological parameter for comparison in subsequent health monitoring for the convenience of the user to learn an evolution process of physical status of the monitored object, particularly the disease variation trend of the patient, so as to further develop or change the treatment plan accordingly.

In one embodiment of the health monitoring television according to the present disclosure, the central processor is configured to perform comparative analysis to the acquired physiological parameter and the stored corresponding normal physiological parameter value and take the result of the comparative analysis as a portion of the health monitoring information. Thus, it is benefit for the user to further learn the exact and detailed physical status of the monitored object, such as whether it is at the normal level.

In one embodiment of the health monitoring television according to the present disclosure, the central processor is further configured to grade the physical status of the monitored object in terms of health level based on the comparative analysis result and give a corresponding processing suggestion, and these information is taken as a portion of the health monitoring information. Thus, the user can adopt corresponding countermeasures with respect to different conditions while learning the health level of the monitored object.

In one embodiment of the health monitoring television according to the present disclosure, it further comprises a network interface for connecting with a network server so as to transmit the health monitoring information to a network database, thereby facilitating the doctor or the family member to learn the physical status of the aged at home in real time at the display terminal (mobile phone or computer) through the network.

In one embodiment of the health monitoring television according to the present disclosure, it further comprises a camera having a facial recognition function, for recognizing different monitored objects so as to perform monitoring to them respectively, thereby avoiding confusion of different objects' data.

In one embodiment of the health monitoring television according to the present disclosure, it further comprises an instruction input device, wherein the central processor is further configured to control the health monitoring television based on an instruction inputted by the user via the instruction input device, so as to facilitate the user to control the health monitoring process of the television depending on his or her own conditions or preference.

In one embodiment of the health monitoring television according to the present disclosure, the instruction input device comprises at least one of a remote controller and a voice input device. With use of different instruction input devices, the user experience can be enriched and more appropriate instruction input manners can be achieved for different types of monitored objects. For example, the voice input device is more suitable for an object with vision disorder apparently.

In one embodiment of the health monitoring television according to the present disclosure, the physiological parameter acquisition device comprises at least one of a body temperature sensor, a heart rate sensor, an ECG sensor, a blood pressure sensor, a blood oxygen sensor, a blood sugar sensor and a pulse wave sensor. Thus, this is beneficial for the user to learn the physical status of the monitored object comprehensively.

In one embodiment of the health monitoring television according to the present disclosure, the central processor is connected with the physiological parameter acquisition device wirelessly. For example, the wireless connection can comprise Bluetooth, ZigBee, and infrared connection.

According to another aspect of the present disclosure, there is proposed a working method of the health monitoring television according to the present disclosure, comprising the steps of: acquiring a physiological parameter of a monitored object through a physiological parameter acquisition device; generating health monitoring information based on the acquired physiological parameter through a central processor; and outputting the health monitoring information through an information exhibition device. Generally, the health monitoring information can simply comprise original physiological parameter information. The health monitoring information can further comprise the comparison result between the current physiological parameter value and the normal value, so as to reflect the physical status more directly and specifically. Or, the health monitoring information can further comprise the comparison result of the current physiological parameter value with the previous physiological parameter value of the same monitored object, for the convenience of the user (such as the monitored object, or his or her family member or doctor) to learn the evolution condition of the physical status of the monitored object.

In one embodiment of the working method of the health monitoring television according to the present disclosure, the step of generating the health monitoring information can further comprise: grading physical status of the monitored object based on the above comparison result and giving a corresponding suggestion, so that the health monitoring information can comprise grading information and suggestion information.

In one embodiment of the working method of the health monitoring television according to the present disclosure, the grading information can comprise: a normal state and an abnormal state.

In one embodiment of the working method of the health monitoring television according to the present disclosure, the abnormal state comprises a plurality of levels in an ascending order.

In one embodiment of the working method of the health monitoring television according to the present disclosure, when the grading information is determined as an abnormal state for the first time, the abnormal state is defined as level one of the abnormal state, and the method further comprises: determining the grading information repeatedly at a predetermined interval, wherein if it stays in the abnormal state for a predetermined number of times, the abnormal state level will rise up by one automatically, and so on, until the abnormal state level reaches the highest level or the grading information becomes the normal state;
when the grading information becomes the normal state, the health monitoring information comprises a message of out of abnormal state; and
when the abnormal state reaches the highest level, the health monitoring information comprises an obvious danger sign and alarm information.

In such a solution of processing abnormal conditions using dynamic grading, a less dangerous situation (e.g., the condition of level one that can be relieved by self-medication) can be processed more appropriately on the premise of not delaying the rescue of the critically ill monitored object, so as to avoid wasting more public medical resources and time.

### BRIEF DESCRIPTION OF THE DRAWINGS

The various aspects, characteristics and advantages of the present disclosure will be understood easily based on the following detailed description and drawings, in which:

FIG. 1 schematically shows a hardware structure block diagram of a health monitoring television according to an embodiment of the present disclosure;

FIG. 2 schematically shows a functional module diagram of a health monitoring television according to an embodiment of the present disclosure;

FIG. 3 schematically shows a working flow chart of a health monitoring television according to an embodiment of the present disclosure; and

FIG. 4 schematically shows a working flow chart of a health monitoring television according to another embodiment of the present disclosure

It should be noted that these drawings are merely schematic and illustrative, and are not necessarily drawn to scale.

Reference signs: 101—physiological parameter acquisition device; 102—central processor; 103—information exhibition device; 104—memory; 105—network interface; 106—camera; 107—instruction input device; 201—acquisition module; 202—parameter analysis and information generation module; 203—exhibition module; 204—storage module; 205—network module; 206—recognition module; 207—input module.

### DETAILED DESCRIPTION OF THE INVENTION

The present disclosure will be described more completely with reference to the drawings thereinafter.

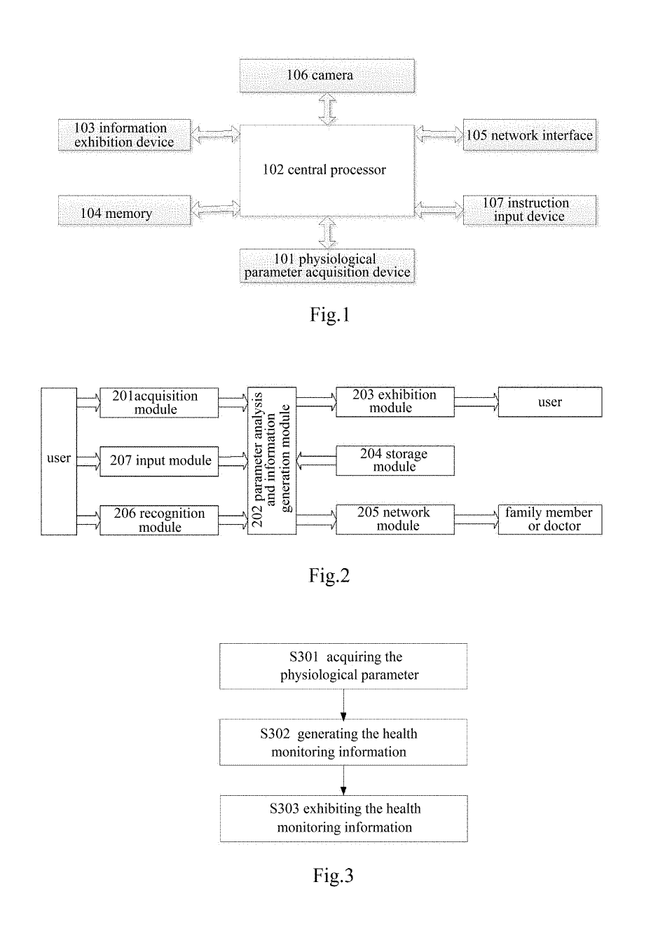

FIG. 1 schematically shows a hardware structure block diagram of a health monitoring television 100 according to an embodiment of the present disclosure. The health monitoring television 100 comprises a physiological parameter acquisition device 101 for acquiring a physiological parameter of a monitored object; a central processor 102 for generating health monitoring information based on the acquired physiological parameter; and an information exhibition device 103 for outputting the health monitoring information to a user. The information exhibition device 103 can be a television screen and/or a television speaker. Correspondingly, the health monitoring information can be outputted to the user in the form of video and/or audio. In this way, the user can view or listen to the health monitoring information easily so as to learn the physical status thereof.

Generally, the physiological parameter acquisition device 101 as shown in FIG. 1 can be any sensor that can sense the various physiological parameters of a human body (such as body temperature, heart rate, blood pressure etc.). Consequently, the physiological parameter acquisition device 101 can comprise one or more of for example a body temperature sensor, a heart rate sensor, an ECG sensor, a blood pressure sensor, a blood oxygen sensor, a blood sugar sensor and a pulse wave sensor and the like. These sensors can use the wearable sensors or portable sensors of the prior art.

In addition, the physiological parameter acquisition device 101 can establish communication with the health monitoring television host through physical connection, for example, connecting with the central processor through cables for signal transmission; or can also connect with the television host (particularly the central processor) wirelessly. Generally, the wireless connection is more appropriate for communication, because the monitored object must be fixed at a certain position and cannot move when using the wired connection, while the monitored object mounted with a wireless sensor can move freely within a certain range, which has less influence on the daily life of the user so as to optimize the user experience significantly. The wireless connection between the above wireless sensor and the central processor 102 can use various commonly used short-range wireless communication technologies including near field communication (NFC), Bluetooth, ZigBee, infrared, Wi-Fi etc.

As shown in FIG. 1, the central processor 102 is the core of the whole hardware structure of the health monitoring television, and is the center of the whole system operation for information transceiving, analysis and processing and control of various hardwares or functional modules (such as interrupting or triggering a certain functional module). The information processing for example includes necessary signal processing, such as a series of signal processing of amplifying, filtering, compressing etc., to the data transmitted to it, so as to facilitate further analysis and use.

As shown in FIG. 1, the health monitoring television according to embodiments of the present disclosure can further comprise a memory 104 for storing a normal physiological parameter value corresponding to the acquired physiological parameter, for being compared with the acquired physiological parameter value of the monitored object by the central processor 102, so as to generate health monitoring information that can reflect the physical status of the monitored object more directly. Moreover, the memory 104 can be further used for storing the acquired physiological parameter value of the monitored object for comparison in the subsequent health monitoring so as to learn the evolution process of the physical status of the monitored object. Further, the comparison result or the generated health monitoring information can also be stored in this memory so as to provide necessary physical status information for subsequent possible diagnosis and treatment. Here, the memory 104 can use any information recording mediums, for example, a flash memory device, a RAM, a ROM, a removable memory, a CD-ROM, a hard disk driver, a DVD, a floppy disk or a memory card etc.

As shown in FIG. 1, the health monitoring television according to embodiments of the present disclosure can further comprise a network interface 105 for connecting the health monitoring television to the network so as to transmit the related physiological parameter data and health monitoring information of the monitored object to the database of the network server, so that other people (such as family members, doctors etc.) who are not around the monitored object can learn at any time or monitor in real time the physical status information of the monitored object through terminal devices such as a networked computer or a smart mobile phone etc. The above network can include wide area network (such as Internet), local area network, client-server network (e.g., cloud computing network) etc. Correspondingly, the network interface 105 as shown in FIG. 1 can be any wired or wireless network interface, and the wireless network interface can for example be connected with the preinstalled wireless router at home through Wi-Fi so as to be connected to the Internet.

Further, with respect to multi-user cases (for example, multiple members' health in the family need to be monitored), as shown in FIG. 1, the health monitoring television according to embodiments of the present disclosure can further comprise a camera 106 with a facial recognition function, for recognizing and distinguishing different monitored objects. Only in this way can the measurement data of different monitored objects be differentiated, so as to display the health monitoring information of different users accurately and avoid confusion. The camera 106 can be integrated on the health monitoring television host or can also be integrated on the physiological parameter acquisition device 101, for capturing the facial information at any time for identity recognition and subsequently transmitting the recognition information to the central processor 102. The central processor 102 binds the identity recognition information to the corresponding acquired data, and in this way, when the corresponding health monitoring information is obtained and displayed via signal analysis process, the identity information is also included therein so as to avoid confusion of the information of different monitored objects.

As shown in FIG. 1, the health monitoring television according to embodiments of the present disclosure can further comprise an instruction input device 107 for controlling the health monitoring television based on the instruction inputted by the user, particularly controlling the health monitoring function thereof. Such an instruction input device 107 can comprise for example a manual instruction input device or a voice command input device etc., wherein the manual instruction input device can be a TV remote controller with buttons or keyboard, while the voice command input device can be a microphone integrated on the health monitoring television host. In particular, besides the conventional buttons for remote control of the television functions, various buttons for controlling the health monitoring function can be arranged on the TV remote controller, such as triggering and/or interrupting acquisition of the physiological parameter, analysis processing or information display etc. In addition, some special function buttons can also be arranged on the remote controller, such as a timing detection button, and by pressing this button, the health monitoring television can be triggered to turn on the health monitoring function at regular time automatically until this button is pressed again.

FIG. 2 schematically shows a functional module diagram of a health monitoring television according to an embodiment of the present disclosure, the arrows therein represent the directions of the signal flows. As shown in FIG. 2, the health monitoring television according to an embodiment of the present disclosure comprises a plurality of functional modules corresponding to various hardwares as shown in FIG. 1, i.e., a physiological parameter acquisition module 201, a physiological parameter analysis and health monitoring information generation module 202, a health monitoring data exhibition module 203, a storage module 204, a network communication module 205, a facial recognition module 206 and an instruction input module 207. As shown in FIG. 2, during working of the health monitoring television, firstly, the parameter acquisition module 201 acquires the user's physiological parameter and the facial recognition module 206 recognizes the user facial information, and the user can also input instruction information through the instruction input module 207. Subsequently, the parameter analysis and information generation module 203 analyzes and processes the physiological parameter data obtained from the user based on the instruction information and the recognition information, so as to generate the health monitoring information. Finally, the health monitoring information is exhibited to the user through the information exhibition module 203 such as the television screen or the speaker, or is communicated to the family member or the doctor through the network communication module 205. Here, the obtained health monitoring information can comprise the above physical status information obtained via comparative analysis and the corresponding processing suggestion. Thus, the user, particularly the aged and the patients, can make use of the respective functional modules 201-207 of the health monitoring television according to the present disclosure to learn the physical status thereof and the corresponding countermeasures easily, which solves the problems of complicated operation and inconvenience in use in the prior art, and improves the user experience at the same time.

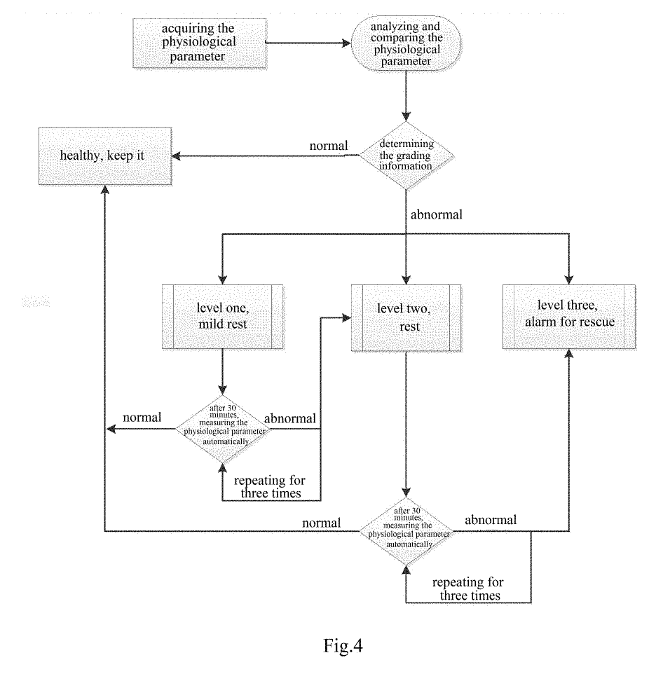

FIG. 3 schematically shows a working method flow chart of a health monitoring television according to an embodiment of the present disclosure. As shown in FIG. 3, the working method of the health monitoring television according to an embodiment of the present disclosure comprises the steps of: S301, the physiological parameter acquisition device 101 acquires the physiological parameter of the monitored object; S302, the central processor 102 generates the health monitoring information based on the acquired physiological parameter; and S303, the information exhibition device 103 outputs the health monitoring information. Generally, the health monitoring information can simply comprise the original physiological parameter information. The health monitoring information can further comprise a comparison result between the current physiological parameter value and the normal value, so as to reflect the physical status more directly and specifically. Or, the health monitoring information can further comprise a comparison result of the current physiological parameter value with the previous physiological parameter value of the same monitored object, for the convenience of the user (such as the monitored object or the family member thereof or the doctor) to learn the evolution condition of the physical status of the monitored object. In this way, for chronic patients for example, the doctor or the family member can make or change the treatment plan timely based on the above evolution condition of the physical status. For example, if the physical status improves, the existing treatment plan can still be used; however, if the physical status becomes worse or is deteriorated, the treatment plan has to be changed timely.

In one embodiment of the working method of the health monitoring television according to the present disclosure, the step of generating the health monitoring information can further comprise grading the physical status of the monitored object based on the above comparison result and giving a corresponding suggestion, so that the health monitoring information can comprise grading information and suggestion information.

For example, the physical status can be graded based on a certain grading standard (such as the physiological parameter threshold) or the result is assessed preliminarily and suggestions are given with reference to the medical knowledge. In particular, for example, two thresholds can be set, which are taken as the limits to divide the parameter values into three intervals, the parameter value that falls within an interval corresponds to its respective grading information. For example, assume that the physiological parameter is the body temperature, two body temperature thresholds can be set, i.e.: t1=37.2° C. and t2=38° C., and when the measured body temperature value t<=t1, it is normal and is suggested be kept it; when t2=>t>t1, it is low fever and the monitored object is suggested to take physical cooling and drink more water; however, when t>t2, it is high fever and it is suggested to take fever medicine and/or go to a doctor timely. Certainly, it should be noted that the above example is given only for the purpose of explanation rather than limitation to the present disclosure. In fact, the grading standard of the physical status is often not the same for different types of monitored objects. For example, compared to young people, the blood pressure of the aged is generally higher, hence, when the grading standard is set, the above factor should be considered appropriately so as to slightly upgrade the grading standard (such as the blood pressure threshold) of the aged.

FIG. 4 schematically shows a working method flow chart of the health monitoring television according to another embodiment of the present disclosure. Compared to FIG. 3, the working method of the health monitoring television as shown in FIG. 4 further comprises the steps of performing comparative analysis and performing grading, as well as different processing measures performed with respect to different grading information. As shown in FIG. 4, the grading information for example can comprise: a normal state and an abnormal state, and the corresponding suggestions can respectively be: continue to keep it, take medicine or go to a doctor and take quick acting drugs and/or alarm for rescue. It should be noted that the above grading information can also use other grading schemes, for example, being more finely divided into healthy, sub-healthy, unhealthy and dangerous states, and more accurate and reasonable suggestions and measures can be given by combining the medical knowledge.

The abnormal state mentioned above can comprise a plurality of levels in an ascending order, as shown in FIG. 4, which can for example comprise level one, level two and level three. When the grading information is determined as the abnormal state for the first time, the abnormal state is defined as level one of the abnormal state. When the grading information is at level one of the abnormal state, the television screen or speaker of the health television prompts the monitored object in the form of video (such as yellow prompt block) or voice to take mild rest and take the corresponding medicine for treatment. And the above comparative analysis and grading step can be repeated at a predetermined time interval (e.g., every 30 minutes) for a predetermined number of times, e.g. three times. If the grading information after three times of repetition is still the abnormal state, the abnormal state will be changed to level two of the abnormal state; but if the grading information is changed to the normal state, the information exhibition device will output a message of out of abnormal state.

When the abnormal state is at level two of the abnormal state, the health monitoring television prompts in a more obvious way (e.g., red prompt block) the monitored object to go to bed and have a rest and take corresponding medicine to relieve the symptom, meanwhile, the comparative analysis and the grading step can be repeated for a predetermined number of times at a predetermined time interval (e.g., every 30 minutes) for a predetermined number of times, e.g., 3 times. If the grading information after three times of repetition is still the abnormal state, the abnormal state will be changed to level three of the abnormal state; but if the grading information is changed to the normal state, the information exhibition device will output the message of out of abnormal state.

When the abnormal state is at level three, the health monitoring television will not display the TV programs and will exhibit obvious danger signs and alarm information through the information exhibition device. For example, the television screen prompts successively with danger signs, and the volume of the television speaker is adjusted to the maximum automatically to arouse the attention of neighbors in the form of call for help. Meanwhile, a one-key alarm function preset on the remote controller can be triggered. The monitoring station or hospital as well as the family members can receive over the network the information that the monitored object is in the extremely dangerous state, so as to urge the emergency doctor and/or the family member to rush to the scene to rescue.

In the above embodiment, the abnormal state is further divided into for example three levels: level one, level two and level three. This division is made according to whether the medicine can restrain the abnormal state as well as the effect time thereof, which aims to using such a dynamic grading to process the abnormal condition in the event that the danger degree cannot be determined. In this way, a less dangerous situation (e.g., the condition of level one or level two that can be relieved by self-medication) can be processed more appropriately on the premise of not delaying the rescue of the critically ill monitored object, so as to avoid wasting more public medical resources and time.

Certainly, according to different types of monitored objects and the actual conditions thereof, the abnormal state as shown in FIG. 4 can also be graded according to other standards or not be graded. For example, for some extremely dangerous sudden illness, such as acute myocardial infarction, cerebral hemorrhage etc., if the corresponding physiological parameter is abnormal, an alarm must be issued immediately and the patient must be sent to the hospital for rescue, instead of the grading processing as shown in FIG. 4, otherwise, it will cause a serious problem.

Although the particular embodiments of the present disclosure have been shown and described, for the skilled person in the art, however, several modifications and variations can be made in a wider aspect thereof without departing from the present disclosure. Therefore, the appended Claims should encompass all of such modifications and variations within the scope thereof, which is similar to falling within the real spirit and scope of the present disclosure.

1. A health monitoring television, comprising:
a physiological parameter acquisition device for acquiring a physiological parameter of a monitored object;
a central processor for generating health monitoring information based on the acquired physiological parameter; and
an information exhibition device for outputting the health monitoring information to a user.

2. The health monitoring television according to claim 1, wherein the information exhibition device comprises at least one of a television screen and a television speaker, and the health monitoring information comprises the acquired physiological parameter.

3. The health monitoring television according to claim 2, further comprising a memory for storing a normal physiological parameter value corresponding to the acquired physiological parameter, wherein the central processor is configured to perform comparative analysis to the acquired physiological parameter and the stored corresponding normal physiological parameter value and take a result of the comparative analysis as a portion of the health monitoring information.

4. The health monitoring television according to claim 3, wherein the memory is further used for storing the acquired physiological parameter for comparison in subsequent health monitoring so as to learn an evolution process of physical status of the monitored object.

5. The health monitoring television according to claim 3, wherein the central processor is further configured to grade the physical status of the monitored object based on the comparative analysis result and give a corresponding processing suggestion, and the health monitoring information comprises the grading and processing suggestion information.

6. The health monitoring television according to claim 3, further comprising a network interface for connecting with a network server so as to transmit the health monitoring information to a network database.

7. The health monitoring television according to claim 3, further comprising a camera having a facial recognition function, for recognizing different monitored objects so as to perform monitoring to them respectively.

8. The health monitoring television according to claim 2, further comprising an instruction input device, wherein the central processor is further configured to control the health monitoring television based on an instruction inputted by the user via the instruction input device.

9. The health monitoring television according to claim 8, wherein the instruction input device comprises at least one of a remote controller and a voice input device.

10. The health monitoring television according to claim 1, wherein the physiological parameter acquisition device comprises at least one of a body temperature sensor, a heart rate sensor, an ECG sensor, a blood pressure sensor, a blood oxygen sensor, a blood sugar sensor and a pulse wave sensor.

11. The health monitoring television according to claim 1, wherein the central processor is wirelessly connected with the physiological parameter acquisition device.

12. The health monitoring television according to claim 11, wherein the wireless connection comprises Bluetooth, ZigBee, infrared connection, WIFI.

13. A working method of the health monitoring television according to claim 1, comprising the steps of:
acquiring a physiological parameter of a monitored object through the physiological parameter acquisition device;
generating health monitoring information based on the acquired physiological parameter through the central processor; and
outputting the health monitoring information through the information exhibition device.

14. The method according to claim 13, wherein the step of generating the health monitoring information comprises: performing comparative analysis to the acquired physiological parameter and a corresponding normal physiological parameter value stored in a memory in advance, so as to enable the health monitoring information to comprise a comparative analysis result.

15. The method according to claim 14, wherein the step of generating the health monitoring information further comprises: grading the physical status of the monitored object based on the comparative analysis result and giving a corresponding suggestion, so as to enable the health monitoring information to comprise grading information and suggestion information.

16. The method according to claim 15, wherein the grading information comprises: a normal state and an abnormal state.

17. The method according to claim 16, wherein the abnormal state comprises a plurality of levels in an ascending order.

18. The method according to claim 16, wherein when the grading information is determined as an abnormal state for the first time, the abnormal state is defined as level one of the abnormal state, and the method further comprises: determining the grading information repeatedly at a predetermined time interval, and if it stays in the abnormal state for a predetermined number of times, the abnormal state level will rise up by one automatically, and so on, until the abnormal state level reaches the highest level or the grading information becomes the normal state; and
when the grading information becomes the normal state, the health monitoring information comprises a message of out of abnormal state;
when the abnormal state reaches the highest level, the health monitoring information comprises obvious danger signs and alarm information.

19. The health monitoring television according to claim 5, further comprising a network interface for connecting with a network server so as to transmit the health monitoring information to a network database.

20. The health monitoring television according to claim 5, further comprising a camera having a facial recognition function, for recognizing different monitored objects so as to perform monitoring to them respectively.
